(19) United States
(12) Patent Application Publication
(10) Pub. No.: US 2022/0296561 A1
(43) Pub. Date: Sep. 22, 2022

(54) NON-ORAL MEDICATED CANNABINOID COMPOSITIONS, METHODS OF MANUFACTURING, AND METHODS OF TREATMENT

(71) Applicant: Tauriga Sciences Inc., Wappingers Falls, NY (US)

(72) Inventor: Seth Marcus Shaw, Wappingers Falls, NY (US)

(21) Appl. No.: 17/693,609

(22) Filed: Mar. 14, 2022

Related U.S. Application Data
(60) Provisional application No. 63/162,100, filed on Mar. 17, 2021.

(51) Int. Cl. A61K 31/352; A61K 31/12; A61K 31/05; A61K 9/02; A61K 9/00; A61K 47/42; A61K 47/10; A61K 47/32; A61K 47/12

(52) U.S. Cl. CPC A61K 31/352; A61K 31/12; A61K 31/05; A61K 9/02; A61K 9/0014; A61K 47/42; A61K 47/10; A61K 47/32; A61K 47/12

(57) ABSTRACT

Disclosed in certain embodiments is a composition for treating nausea and/or vomiting. The composition could be in any form suitable for non-oral administration. The composition may include at least one cannabinoid components, such as cannabidiol and/or cannabigerol, and a ginger component. The composition may include any one of these components or any combination of these components in an effective amount to treat nausea and/or vomiting. Also disclosed herein are methods of preparing a composition to treat nausea and/or vomiting and methods of treating nausea and/or vomiting.

### CROSS REFERENCE TO RELATED APPLICATIONS

The present application claims priority to U.S. Provisional Patent Application No. 63/162,100 filed on Mar. 17, 2021, the entire contents of which are incorporated herein.

### FIELD OF THE INVENTION

In certain embodiments, the present invention relates to the field of pharmaceutical compositions for treating nausea and/or vomiting, methods of preparation thereof, and methods for treating nausea and/or vomiting.

### BACKGROUND OF THE INVENTION

Cancer treatments, such as chemotherapy and radiation therapy, can cause nausea and vomiting. Some drugs, such as targeted therapy and immunotherapy, can also cause nausea and vomiting. Some types of cancers may also contribute to nausea and vomiting. Nausea and/or vomiting may also be triggered due to peripheral factors (such as substance intoxication), due to psychological factors (such as anxiety), under post-operative circumstances, due to pregnancy, due to motion sickness, and the like.

Individuals are often provided medicines to prevent nausea and vomiting from starting and/or to manage and/or treat the nausea and vomiting when it begins. Various types of anti-emetic drugs are currently available for a variety of types of nausea and vomiting. Some examples of such drugs include, serotonin (5-HT3) antagonists for acute nausea, NK-1 receptor antagonists for delayed nausea, steroids, dopamine antagonists, and olanzapine, to name a few.

Cannabis contains many chemical compounds useful for medicinal or recreational applications due to their high concentration of cannabinoids. Cannabinoids may also assist with preventing and/or treating nausea and/or vomiting experienced by oncology subjects. Cannabinoids may also assist with preventing and/or treating nausea and/or vomiting triggered by various other reasons.

Cannabis is a complex plant with over 400 chemical entities of which more than 60 of them are cannabinoid compounds, some of them with opposing effects. [See: Cannabis, a complex plant: different compounds and different effects on subjects. Zerrin Atakan, Ther Adv Psychopharmacol. 2012 December; 2(6): 241-254. doi: 10.1177/2045125312457586 and references therein; Downloaded Mar. 8, 2021 from https://www.ncbi.nlm.nih.gov/pmc/articles/PMC3736954/]

The cannabinoid family of compounds include such compounds as delta-9-THC, delta-8-THC, cannabinol, cannabidiol, delta-9-tetrahydrocannabivarin, cannabigerol and cannabichromene.

There exists a need in the art for a pharmaceutical composition for treating nausea and/or vomiting in a subject, a method for preparing said pharmaceutical composition, and a method for treating nausea and/or vomiting in a subject in need thereof.

### OBJECTS AND SUMMARY OF THE INVENTION

It is an object of certain embodiments of the present invention to provide a pharmaceutical composition (e.g., a non-oral composition suitable for intranasal administration, parenteral administration, transdermal administration, rectal or vaginal administration, ocular administration, otic administration, and the like) for preventing nausea in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a pharmaceutical composition (e.g., a non-oral composition suitable for intranasal administration, parenteral administration, transdermal administration, rectal or vaginal administration, ocular administration, otic administration, and the like) for treating nausea in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a pharmaceutical composition (e.g., a non-oral composition suitable for intranasal administration, parenteral administration, transdermal administration, rectal or vaginal administration, ocular administration, otic administration, and the like) for preventing vomiting in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a pharmaceutical composition (e.g., a non-oral composition suitable for intranasal administration, parenteral administration, transdermal administration, rectal or vaginal administration, ocular administration, otic administration, and the like) for treating vomiting in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a method for treating nausea in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a method for treating vomiting in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a method for preventing nausea in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a method for preventing vomiting in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

It is an object of certain embodiments of the present invention to provide a method for manufacturing a pharmaceutical composition suitable for treatment and/or prevention of nausea and/or vomiting in a subject in need thereof (e.g., a cancer subject, a person experiencing motion sickness, a person experiencing substance intoxication, pregnant individuals, a person that has undergone an operation and experiences post-operative nausea and/or vomiting symptoms, or the like).

The above objects and others may be achieved by the present invention which in certain embodiments is directed to a pharmaceutical composition (e.g., a non-oral composition suitable for intranasal administration, parenteral administration, transdermal administration, rectal or vaginal administration, ocular administration, otic administration, and the like) for providing nausea and/or vomiting treatment to a subject in need thereof, or for preventing nausea and/or vomiting in a subject in need thereof, wherein the pharmaceutical composition includes one or more cannabinoid components in an effective amount to treat nausea and/or vomiting and a ginger component(s). In certain embodiments, the pharmaceutical composition includes one or more cannabinoid components in an effective amount to treat nausea and/or vomiting, a ginger component(s), and a pharmaceutically acceptable excipient, wherein the pharmaceutical composition is formulated for non-oral administration.

In certain embodiments, the pharmaceutical compositions described herein refer to a non-oral composition (e.g., a non-oral composition suitable for intranasal administration, parenteral administration, transdermal administration, rectal or vaginal administration, ocular administration, otic administration, and the like) that includes one or more cannabinoid components and a ginger component(s), wherein the one or more cannabinoid components and the ginger component(s), together, are present in the non-oral composition in an effective amount to treat and/or prevent nausea and/or vomiting in a subject in need thereof, wherein the pharmaceutical composition is formulated for non-oral administration.

In certain embodiments, the present disclosure is directed to a method for treating and/or preventing nausea and/or vomiting in a subject in need thereof. In certain embodiments, the method includes administering to a subject in need thereof any of the non-oral compositions described herein. For instance, in one embodiment, the non-oral composition includes one or more cannabinoid components and a ginger component(s). In another embodiment, the method includes administering the non-oral composition to the subject, wherein the administration may be intranasal administration, parenteral administration, transdermal administration, rectal or vaginal administration, ocular administration, or otic administration.

In certain embodiments, particular subjects that may benefit from the method described herein are cancer patients that could experience nausea and/or vomiting due to any number of reasons, such as, without limitations, the cancer treatment regime, the cancer itself, side effects to other drugs, and so on. Other subjects who could experience nausea and/or vomiting due to any number of reasons, such as, underlying medical conditions, as a post-operative symptom, substance intoxication, motion sickness, pregnancy, psychological factors, and the like, may also benefit from the pharmaceutical compositions described herein.

In certain embodiments, the present disclosure is directed to a method of manufacturing a non-oral pharmaceutical composition (e.g., a non-oral composition suitable for intranasal administration, parenteral administration, transdermal administration, rectal or vaginal administration, ocular administration, otic administration, and the like) that is suitable for treating and/or preventing nausea and/or vomiting in a subject in need thereof. In certain embodiments, the method includes combining the one or more cannabinoid components with the ginger component(s) and a pharmaceutically acceptable excipient to form any of the non-oral compositions described herein.

In certain embodiments, the method may include formulating one or more cannabinoid components with a ginger component(s) into a parenteral formulation (e.g., suitable for administration subcutaneously, intramuscularly, intravenously, or intradermally).

In certain embodiments, the method may include formulating one or more cannabinoid components with a ginger component(s) into a formulation suitable for rectal or vaginal administration (e.g., as a suppository, enema, tablets, pessary, gel, cream, foam, sponge).

In certain embodiments, the method may include formulating one or more cannabinoid components with a ginger component(s) into a formulation suitable for topical or transdermal administration (e.g., as an ointment, cream, lotion, gel, spray, patch).

In certain embodiments, the method may include formulating one or more cannabinoid components with a ginger component(s) into a formulation suitable for nasal administration (e.g., drops, sprays).

In certain embodiments, the method may include formulating one or more cannabinoid components with a ginger component(s) into a formulation suitable for ocular administration (e.g., solutions, emulsions, suspension, ointments, contact lens, implants, inserts, intravitreal).

In certain embodiments, the method may include formulating one or more cannabinoid components with a ginger component(s) into a formulation suitable for otic administration (e.g., topical, intratympanic, intracochlear).

In certain embodiments, the present disclosure is directed to a kit that includes a container and any of the non-oral compositions described herein stored within the container. The container may be a bottle, a bag, a blister package, a tube, or any other suitable packaging for any of the non-oral compositions described herein. In certain embodiments, the container may include a delivery device, such as, a spray delivery device, an aerosol delivery device, a device for assisting with suppository placement, and the like. In certain embodiments, the pharmaceutical compositions may be packaged in single dose containers/packages.

In certain embodiments, the one or more cannabinoid components includes, without limitations, cannabidiol, cannabigerol, tetrahydrocannabinol, cannabinol, cannabichromene, or a combination thereof. In one embodiment, the non-oral composition includes a combination of cannabidiol and cannabigerol.

In certain embodiments, the ginger component(s) includes, without limitations, ginger, a ginger root extract, a shogaol, a zingerone, a gingerol, or a combination thereof. In one embodiment, the non-oral composition includes one or more types of gingerol as the ginger component(s).

In certain embodiments, the cannabidiol is present in any of the non-oral pharmaceutical compositions described herein in a therapeutically effective amount to treat and/or prevent nausea and/or vomiting in a subject in need thereof. In certain embodiments, the cannabigerol is present in any of the non-oral pharmaceutical compositions described herein in a therapeutically effective amount to treat and/or prevent nausea and/or vomiting in a subject in need thereof. In certain embodiments, the gingerol is present in any of the non-oral pharmaceutical compositions described herein in a therapeutically effective amount to treat and/or prevent nausea and/or vomiting in a subject in need thereof. In certain embodiments, two or more of the cannabidiol, cannabigerol, and gingerol are, together, present in any of the non-oral pharmaceutical compositions described herein in a therapeutically effective amount to treat and/or prevent nausea and/or vomiting in a subject in need thereof.

In certain embodiments, the cannabidiol is the sole active agent in the non-oral compositions described herein. In certain embodiments, the cannabigerol is the sole active agent in the non-oral compositions described herein. In certain embodiments, the gingerol is the sole active agent in the non-oral compositions described herein. In certain embodiments, two or more of the cannabidiol, cannabigerol, and gingerol are, together, the active agents in the non-oral compositions described herein.

### Definitions

As used herein, the singular forms “a,” “an,” and “the” include plural references unless the context clearly indicates otherwise. Thus, for example, reference to “an antioxidant” includes a single antioxidant as well as a mixture of two or more different antioxidants; reference to “a ginger component” includes a single ginger component as well as a mixture of two or more different ginger components; reference to “an excipient” includes a single excipient as well as a mixture of two or more different excipients; reference to “a cannabinoid component” includes a single cannabinoid component as well as a mixture of two or more different cannabinoid components; and the like.

As used herein, the term “about” in connection with a measured quantity, refers to the normal variations in that measured quantity, as expected by one of ordinary skill in the art in making the measurement and exercising a level of care commensurate with the objective of measurement. In certain embodiments, the term “about” includes the recited number ±10%, such that “about 10” would include from 9 to 11.

As used herein, the term “active agent” or “active ingredient” refers to any material that is intended to produce a therapeutic, prophylactic, or other intended effect, whether or not approved by a government agency for that purpose. In certain embodiments, active agents may be provided in either powder or oil form. In certain embodiments, an active in oil form is defined as a free flowing liquid, semi-solid, or paste that is lipid-based and not water soluble. In certain embodiments, when a semi-solid or paste active agent is heated to a maximum temperature of 140 degree F., the material changes to an oil. In certain embodiments, active ingredients in oil form can include hemp oil, THC (delta-9-tetrahydrocannabinol) resin, any cannabinoid oil, as well as pharmaceutical actives, botanicals and essential oils.

This term with respect to a specific agent includes the pharmaceutically active agent, and all pharmaceutically acceptable salts, solvates and crystalline forms thereof, where the salts, solvates and crystalline forms are pharmaceutically active. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

The term “solvate” refers to an aggregate that comprises one or more molecules of active agent with one or more molecules of a solvent. The solvent may be water, in which case the solvate may be a hydrate. Alternatively, the solvent may be an organic solvent. In one embodiment, “solvate” refers to the active pharmaceutical ingredient in its state prior to dissolution. Alternatively, the solid particles of a suspended active agent may comprise a co-precipitated solvent.

As used herein, the phrase “pharmaceutically acceptable” means that which is useful in preparing a pharmaceutical composition that is generally safe, non-toxic, and is not biologically or otherwise undesirable and is acceptable for human pharmaceutical use.

As used herein, the terms “therapeutically effective” and an “effective amount” refer to that amount of an active agent or the rate at which it is administered needed to produce a desired therapeutic result, such as to treat and/or prevent a condition or symptoms of a condition in a subject.

The term “subject” refers to a human or animal, who has demonstrated a clinical manifestation of nausea and/or vomiting suggesting the need for a nausea and/or vomiting treatment, or who is at risk of experiencing nausea and/or vomiting. The term “subject” may also refer to a human or an animal, who has demonstrated a clinical manifestation of symptoms that are associated or may be associated with nausea and/or vomiting, or who is at a risk of experiencing such symptoms, suggesting the need for a nausea and/or vomiting treatment. For example, a cancer/oncology subject who experiences nausea and/or vomiting from their existing cancer treatment regime (e.g., cytotoxic chemotherapy and/or radiotherapy) or who is about to start a cancer treatment regime that could cause nausea and/or vomiting and the subject is treated prophylactically with any of the pharmaceutical compositions described herein. The subject in need thereof may include individuals that are experiencing (or may experience) nausea and/or vomiting due to underlying medical conditions, substance intoxication, motion sickness, psychological factors, post operatively, pregnancy, and the like. The subject in need thereof may also include individuals that are experiencing (or may experience) nausea and/or vomiting and/or the symptoms of nausea and/or vomiting due to any other reason, whether explicitly described herein or not. In certain embodiments, a subject in need thereof may be an individual who experiences nausea and/or vomiting related to peripheral factors such as ingestion of toxins (e.g., alcohol/drug intoxication), disturbance of the vestibular system, peritoneal inflammation, bowel obstruction, or any other underlying medical condition. Yet another example may be an individual who experiences nausea and/or vomiting related disorders of delayed gastric emptying as, for example, in diabetes and idiopathic gastroparesis. Yet another example may be an individual who experiences nausea and/or vomiting that may be triggered by anxiety, threatening situation, a situation that is regarded as distasteful by the subject, or hostility (such as a temper tantrum), or any other psychological trigger. In certain embodiments, the subject in need thereof refers to an individual who experiences nausea and/or vomiting that may be induced by cytotoxic chemotherapy and/or radiotherapy, or other treatment regimens (whether oncology related or not). In certain embodiments, subject in need thereof refers to an individual who experiences nausea and/or vomiting that is post-operative and/or which may be attributed to the anesthetic agents and/or the analgesic agents that are administered to the individual. In certain embodiments, “subject” refers to an individual who experiences nausea and/or vomiting related to motion sickness (e.g., sea-sickness, car-sickness, air-sickness). In certain embodiments, the term “subject” may refer to an individual experiencing nausea and/or vomiting that is pregnancy related (e.g., a pregnant individual experiencing morning sickness).

The terms “treatment of” and “treating” include the administration of an active agent(s) with the intent to lessen the severity of a condition.

The terms “prevention of” and “preventing” include the avoidance of the onset of a condition by a prophylactic administration of the active agent.

The term “condition” or “conditions” may refer to those medical conditions commonly recognized as nausea, vomiting, or symptoms thereof, such as dizziness, faintness, dry mouth, diarrhea, fever, abdominal pain, decreased urination, or a combination thereof, which can be treated, mitigated, or prevented by a timely administration to a subject of the non-oral composition described herein (e.g., non-oral compositions suitable for intranasal administration, parenteral administration, transdermal administration, rectal or vaginal administration, ocular administration, otic administration, and the like). In certain embodiments, the nausea or vomiting may be physiological and may be triggered by peripheral factors such as ingestion of toxins (e.g., alcohol/drug intoxication), disturbance of the vestibular system, peritoneal inflammation, bowel obstruction, or other underlying medical conditions. It may also occur in certain medical disorders of delayed gastric emptying as, for example, in diabetes and idiopathic gastroparesis. In certain embodiments, the nausea or vomiting may be psychogenic and may be triggered by anxiety, threatening situation, a situation that is regarded as distasteful by a subject, hostility (such as a temper tantrum), or other psychological conditions. In certain embodiments, the nausea and/or vomiting may be induced by cytotoxic chemotherapy and/or radiotherapy, or other treatment regimens (whether oncology related or not). In certain embodiments, the nausea and/or vomiting may be post-operative which may be attributed to the anesthetic agents and/or the analgesic agents that are administered to the subject. In certain embodiments, the nausea and/or vomiting may be related to motion sickness (e.g., sea-sickness, car sickness, air sickness). In certain embodiments, the nausea and/or vomiting may be pregnancy related (e.g., morning sickness).

As used herein, “oral delivery” or “oral administration” refers to a route of administration wherein the pharmaceutical dosage form is taken through the mouth. Oral administration is a part of enteral administration, which also includes buccal (dissolved inside the cheek), sublabial (dissolved under the lip), and sublingual administration (dissolved under the tongue). The term “non-oral” with respect to the composition or with respect to the administration route refers to any composition or administration route that is not taken through the mouth.

As used herein, “nasal delivery” or “nasal administration” or “intranasal administration” refers to a route of administration wherein the pharmaceutical dosage form is taken to, or through, the nose (e.g., nasal cavity).

As used herein, “dermal delivery” or “dermal administration” or “transdermal administration” refers to a route of administration wherein the pharmaceutical dosage form is taken to, or through, the dermis (i.e., layer of skin between the epidermis (with which it makes up the cutis) and subcutaneous tissues).

As used herein, “parenteral administration” refers to a route of administration wherein the pharmaceutical dosage form is injected, e.g., to the muscle (intramuscular administration), to the vein (intravenous administration), under the skin (subcutaneous administration).

As used herein, “ophthalmic delivery” or “ophthalmic administration” or “ocular administration” refers to a route of administration wherein the pharmaceutical dosage form is taken to, or through, the eye.

As used herein, “otic administration” refers to a route of administration wherein the pharmaceutical dosage form is taken to, or through, the ear.

As used herein, “rectal administration” refers to a route of administration wherein the pharmaceutical dosage form is taken to, or through, the rectum. As used herein, “vaginal administration” refers to a route of administration wherein the pharmaceutical dosage form is taken to, or through, the vagina. As used herein, “urethral administration” refers to a route of administration wherein the pharmaceutical dosage form is taken to, or through, the urethra.

As used herein, “capsule” refers to a solid pharmaceutical dosage form wherein the active (and inactive) ingredient is encapsulated. Encapsulation refers to a range of techniques used to enclose medicines in a relatively stable shell known as a capsule. The two main types of capsules include hard-shelled capsules and soft-shelled capsules. Hard-shelled capsules are typically made using gelatin and contain dry, powdered ingredients or miniature pellets made by, e.g. processes of extrusion or spheronisation. These are made in two halves: a lower-diameter “body” that is filled and then sealed using a higher-diameter “cape”. The second main type of capsules include soft-shelled capsules, primarily used for oils and for active ingredients that are dissolved or suspended in oil. Both of these classes of capsules are made from aqueous solutions of gelling agents like such as animal protein mainly gelatin; and plant polysaccharides or their derivatives like carrageenan and modified forms of starch and cellulose. Other ingredients can be added to the gelling agent solution like plasticizers such as glycerin and/or sorbitol to decrease the capsule's hardness, coloring agents, preservatives, disintegrants, lubricants and surface treatment.

As used herein, “tablet” refers to a pharmaceutical dosage form that includes a mixture of active substances and excipients, usually in powder form, pressed or compacted from a powder into a solid dose. The excipients can include diluents, binders or granulating agents, glidants (flow aids) and lubricants to ensure efficient tableting; disintegrants; and pigments to make the tablets visually attractive. A polymer coating is often applied to make the tablet smoother, to control the release rate of the active ingredient, to make it more resistant to the environment (extending its shelf life), or to enhance the tablet's appearance.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

All references to wt. % throughout the specifications and the claims refer to the weight of the component in reference to the weight of the entire subject composition and may also be designated as w/w, unless explicitly indicated otherwise.

### DETAILED DESCRIPTION

### Pharmaceutical Composition

In certain embodiments, the present disclosure is directed to a composition for treatment of nausea and/or vomiting in a subject in need thereof. A subject in need thereof may be a cancer patient that is experiencing nausea and/or to vomiting or a cancer patient that anticipates experiencing nausea and/or vomiting due to treatment, due to the side effects of the cancer, due to other medicines given for health problems that are not cancer related, bowel slowdown or blockage (obstruction), constipation, imbalance of minerals and salts (electrolytes) in the blood, infections, anxiety, other diseases or illness, or any other cause. The subject in need thereof may include individuals that are experiencing (or may experience) nausea and/or vomiting due to underlying medical conditions, substance intoxication, motion sickness, psychological factors, post operatively, pregnancy, and the like. In certain embodiments, a subject in need thereof may be an individual who experiences (or may experience) nausea and/or vomiting related to peripheral factors such as ingestion of toxins (e.g., alcohol/drug intoxication), disturbance of the vestibular system, peritoneal inflammation, bowel obstruction, or other underlying medical conditions. Yet another example may be an individual who experiences (or may experience) nausea and/or vomiting related disorders of delayed gastric emptying as, for example, in diabetes and idiopathic gastroparesis. Yet another example may be an individual who experiences (or may experience) nausea and/or vomiting that may be triggered by anxiety, threatening situation, a situation that is regarded as distasteful by the subject, hostility (such as a temper tantrum), or other psychological conditions. In certain embodiments, subject in need thereof refers to an individual who experiences (or may experience) nausea and/or vomiting that is post-operative and/or which may be attributed to the anesthetic agents and/or the analgesic agents that are administered to the individual. In certain embodiments, subject refers to an individual who experiences (or may experience) nausea and/or vomiting related to motion sickness (e.g., sea-sickness, car-sickness, air-sickness). In certain embodiments, the term subject in need thereof may refer to an individual experiencing nausea and/or vomiting that is pregnancy related (e.g., a pregnant individual experiencing morning sickness). Other individuals who experience (or may experience) nausea and/or vomiting due to any other reason, whether explicitly stated here or not, could also benefit from the non-oral compositions described herein.

In certain embodiments, the non-oral compositions described herein may be used for treating nausea and/or vomiting at the time that the subject is experiencing them or as prophylactic treatment against anticipatory nausea and/or vomiting. In certain embodiments, the non-oral compositions described herein may be used for treating symptoms associated with nausea and/or vomiting (e.g., during the time that the subject is experiencing the symptoms or as a prophylactic treatment). In certain embodiments, the non-oral compositions described herein may be used to address acute nausea and/or vomiting and/or symptoms associated with either one. In certain embodiments, the non-oral composition described herein may be used for a time period, e.g., chronically, to address chronic, reoccurring, or long lasting nausea and/or vomiting and/or symptoms associated with either one

### Dosage Forms

In certain embodiments, the compositions described herein may be in a form that is suitable for administration via one or more of the following routes: parenteral, rectal, vaginal, topical, transdermal, intranasal, ocular, or otic.

In certain embodiments, the compositions described herein may be in a form that is suitable for parenteral administration, such as, intramuscular administration, subcutaneous administration, intravenous administration, or intradermal administration.

In certain embodiments, the compositions described herein may be in a form that is suitable for rectal or vaginal administration. In certain embodiments, compositions suitable for rectal or vaginal administration may be in the form of one or more of a suppository, tablet, pessary, gel, cream, foam, sponge, powder, solution, enema.

In certain embodiments, the compositions described herein may be in a form that is suitable for topical or transdermal administration. In certain embodiments, compositions suitable for topical or transdermal administration may be in the form of one or more of ointments, creams, lotions, gel, sprays, patches.

In certain embodiments, the compositions described herein may be in a form that is suitable for ocular administration. In certain embodiments, compositions suitable for ocular administration may be in the form of one or more of solutions, emulsions, suspension, ointments, contact lens, implants, inserts, intravitreal.

In certain embodiments, the compositions described herein may be in a form that is suitable for otic administration. In certain embodiments, compositions suitable for otic administration may be in the form of one or more of ointments, creams, lotions, gel, sprays, patches, intratympanic, intracochlear.

In certain embodiments, the compositions described herein may be in a form that is suitable for nasal administration. In certain embodiments, compositions suitable for nasal administration may be in the form of one or more of drops or sprays.

In certain embodiments, the compositions described herein may be in a form of a tablet (e.g., for rectal or vaginal administration), which may be prepared, e.g., via compression, via granulation (e.g., wet granulation or dry granulation), via extrusion, via tableting, via compaction, or a combination thereof. In certain embodiments, the tablet may include one or more of the active agents described herein. In certain embodiments, the tablet may include a plurality of layers (such as a core and shell structure or a core and multi-layer shell structure). When more than one active agent is included in the tablet, the active agents may be dispersed homogenously in various parts of the tablet, in certain embodiments. In certain embodiments, the active agents may be separated in various parts of the tablet (e.g., one active agent may be in the core and another active agent may be in the shell). In certain embodiments, the tablet may be coated (e.g., with a compression shell, spray coated, dip coated, cosmetic coating). In certain embodiments, the tablet may be formulated to attain a target disintegration and/or dissolution profile.

In certain embodiments, the compositions described herein may in a form of a tablet for rectal or vaginal administration, which includes one or more cannabinoid components, a ginger component(s), and pharmaceutically acceptable excipients suitable for forming a tablet, wherein the one or more cannabinoid components and the ginger component(s), individually or together, are present in the tablet in an effective amount to treat, reduce, and/or prevent nausea and/or vomiting.

In certain embodiments, the compositions described herein may in a form of a capsule (e.g., a suppository capsule). In certain embodiments, the capsules may include a shell composition enclosing a fill composition. In certain embodiments, the fill composition of the capsule may be liquid, solid (e.g., tablet, beads, powder, particles within a capsule, mini-tablets), semi-solid, or a combination thereof. In certain embodiments, the shell composition may be animal based (e.g., gelatin) or non-animal based. In certain embodiments, the capsule may be coated (e.g., spray coated, dip coated, or include a shell composition with several layers prepared, e.g., via rotary die). In certain embodiments, the capsule may be sealed (e.g., sealed seamlessly). In certain embodiments, the active agents may be separated in various parts of the capsule (e.g., one active agent may be in the fill in a liquid form and a second active agent may be in the fill in a solid form). In certain embodiments, the active agents may be dispersed homogenously in various parts of the capsule. In certain embodiments, the capsule may be formulated to attain a target disintegration and/or dissolution profile.

In certain embodiments, the compositions described herein may in a form of a capsule for rectal or vaginal administration, which includes one or more cannabinoid components, a ginger component(s), and pharmaceutically acceptable excipients suitable for forming a capsule, wherein the one or more cannabinoid components and the ginger component(s), individually or together, are present in the capsule in an effective amount to treat, reduce, and/or prevent nausea and/or vomiting.

In certain embodiments, spray formulations may be filled into a suitable delivery device suitable for delivering a powder/suspension/dispersion/emulsion/liquid composition. In certain embodiments, the compositions described herein may in a form of a spray suitable for nasal or topical administration, the spray may be similar to those known in the art which includes one or more cannabinoid components, a ginger component(s), and pharmaceutically acceptable excipients suitable for forming a spray, wherein the one or more cannabinoid components and the ginger component(s), individually or together, are present in the spray composition in an effective amount to treat, reduce, and/or prevent nausea and/or vomiting.

In certain embodiments, the compositions described herein may in a form of an ointment, cream, lotion, gel, foam, or a patch for rectal, vaginal, ocular, otic, topical, or transdermal administration, which includes one or more cannabinoid components, a ginger component(s), and pharmaceutically acceptable excipients suitable for forming one or more of the ointment, cream, lotion, gel, foam, or a patch, wherein the one or more cannabinoid components and the ginger component(s), individually or together, are present in the composition in an effective amount to treat, reduce, and/or prevent nausea and/or vomiting.

In certain embodiments, the compositions described herein may in a form of a solution, emulsion, or suspension for parenteral, rectal, vaginal, ocular, or nasal administration, which includes one or more cannabinoid components, a ginger component(s), and pharmaceutically acceptable excipients suitable for forming one or more of the solution, emulsion, or suspension, wherein the one or more cannabinoid components and the ginger component(s), individually or together, are present in the composition in an effective amount to treat, reduce, and/or prevent nausea and/or vomiting.

In certain embodiments, the compositions described herein may be in a form of a solution (e.g., an injectable solution), suspension, emulsion, tablet, suppository, ointment, cream, gel, lotion, spray, patch, foam, pessary, drops, powder, implant, insert, or a combination thereof.

In certain embodiments, the compositions may be pre-filled into single unit dose or into a multiple unit dose packaging (which may or may not be a part of a delivery device).

In certain embodiments, the compositions described herein, which may be in any one or more of the dosage forms contemplated herein, include one or more cannabinoid components, a ginger component(s), and pharmaceutically acceptable excipients suitable for forming a particular dosage form, wherein the one or more cannabinoid components and the ginger component(s), individually or together, are present in the non-oral composition in an effective amount to treat, reduce, and/or prevent nausea and/or vomiting.

### Compositions

In certain embodiments, the pharmaceutical compositions described herein may include one or more cannabinoid components. Each of the one or more cannabinoid components may be individually present in the non-oral composition in a therapeutically effective amount to treat and/or prevent nausea and/or vomiting. In certain embodiments, all the cannabinoid components, together, are present in the non-oral composition in a therapeutically effective amount to treat and/or prevent nausea and/or vomiting.

In certain embodiments, the amount of the one or more cannabinoid components, together, in a single dose of the non-oral composition ranges from any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg or about 70 mg to any of about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

In certain embodiments, cannabinoid components that may be included in the non-oral compositions described herein include cannabidiol, cannabigerol, tetrahydrocannabinol, cannabinol, cannabichromene, or a combination thereof. In certain embodiments, the cannabinoid component in the non-oral compositions described herein comprise cannabidiol as the sole active ingredient. In certain embodiments, the cannabinoid component in the non-oral compositions described herein comprise cannabigerol as the sole active ingredient. In certain embodiments, the cannabinoid component in the non-oral compositions described herein comprise a combination of cannabidiol and cannabigerol as the active ingredients.

In embodiments where the non-oral compositions described herein comprises a combination of cannabidiol and cannabigerol, the weight to weight ratio of cannabidiol to cannabigerol ranges from any of about 15:1, about 14:1, about 13:1, about 12:1, about 10:1, about 9:1, or about 8:1 to any of about 7:1, about 6:1, about 5:1, about 4:1, about 3:1, about 2:1, or about 1:1. In certain embodiments, the weight to weight ratio of cannabidiol to cannabigerol ranges from about 15:1 to about 1:1. In certain embodiments, the weight to weight ratio of cannabidiol to cannabigerol ranges from about 12:1 to about 2:1. In certain embodiments, the weight to weight ratio of cannabidiol to cannabigerol ranges from about 10:1 to about 5:1. In certain embodiments, the weight to weight ratio of cannabidiol to cannabigerol is about 8:1.

In certain embodiments, the cannabidiol is present in the non-oral compositions described herein in a therapeutically effective amount to treat and/or prevent nausea and/or vomiting. In certain embodiments, the amount of cannabidiol in a single dose of the non-oral compositions described herein ranges from any of about 10 mg, about 20 mg, about 30 mg, about 40 mg, about 50 mg, about 60 mg, or about 70 mg to any of about 80 mg, about 90 mg, about 100 mg, about 110 mg, about 120 mg, about 130 mg, about 140 mg, or about 150 mg.

In one embodiment, the amount of cannabidiol in a single dose of the non-oral compositions described herein ranges from about 10 mg to about 150 mg. In one embodiment, the amount of cannabidiol in a single dose of the non-oral compositions described herein ranges from about 40 mg to about 120 mg. In one embodiment, the amount of cannabidiol in a single dose of the non-oral compositions described herein ranges from about 70 mg to about 90 mg.

In certain embodiments, the cannabigerol is present in the non-oral compositions described herein in a therapeutically effective amount to treat and/or prevent nausea and/or vomiting. In certain embodiments, the amount of cannabigerol in a single dose of the non-oral compositions described herein ranges from any of about 1 mg, about 2 mg, about 3 mg, about 4 mg, about 5 mg, about 6 mg, or about 7 mg to any of about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg.

In certain embodiments, the amount of cannabigerol in a single dose of the non-oral compositions described herein ranges from any of about 1 mg to about 20 mg. In certain embodiments, the amount of cannabigerol in a single dose of the non-oral compositions described herein ranges from any of about 4 mg to about 15 mg. In certain embodiments, the amount of cannabigerol in a single dose of the non-oral compositions described herein ranges from any of about 8 mg to about 12 mg.

In certain embodiments, the cannabidiol and the cannabigerol are present together in the non-oral compositions described herein in a therapeutically effective amount to treat and/or prevent nausea and/or vomiting. In certain embodiments, the amount of cannabidiol and the cannabigerol together in a single dose of the non-oral compositions described herein ranges from any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg or about 70 mg to any of about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg.

Currently, Cannabis and Cannabis products such as can be obtained in the United States and other countries in various forms. For example, Cannabis can be obtained with a THC (delta-9-tetrahydrocannabinol) level of less than 0.3% by dry weight of material from Cannabis sativa or other varieties of Cannabis and is defined as “hemp”. However, Cannabis sativa and other varieties also exist with a THC content by dry weight of more than 0.3%, and are commonly known as marijuana.

As used herein, “Cannabis” refers to a genus of flowering plants that includes a single species, Cannabis sativa, which is sometimes divided into two additional species, Cannabis indica and Cannabis ruderalis. These three taxa are indigenous to Central Asia, and. South Asia. Cannabis has long been used for fiber (hemp), for seed and seed oils, for medicinal purposes, and as a recreational drug. The Cannabis can include any physical part of the plant material, including, e.g., the leaf, bud, flower, trichome, seed, or combination thereof. Likewise, the Cannabis can include any substance physically derived from Cannabis plant material, such as, e.g., kief and hashish.

As used herein, “leaf” refers to an organ of a vascular plant, as defined in botanical terms, and in particular, in plant morphology. In reference to Cannabis, the first pair of leaves usually have a single leaflet, the number gradually increasing up to a maximum of about thirteen leaflets per leaf (usually seven or nine), depending on variety and growing conditions. At the top of a flowering plant, this number again diminishes to a single leaflet per leaf. The lower leaf pairs usually occur in an opposite leaf arrangement and the upper leaf pairs in an alternate arrangement on the main stem of a mature plant.

As used herein, “bud” refers to a flower-bearing stem or branch of the Cannabis plant, especially a stem or branch bearing a mass of female flowers with associated leaves. The stem or branch bearing the female flowers can be fresh, or can be dried. The pistils of the female Cannabis flower are surrounded by a mass of trichome-rich petals and leaves, and can contain higher concentrations of cannabinoids than do the plant leaves or stems. A bud, e.g., a mass of female flowers and associated leaves, usually covered with trichomes, can be further processed mechanically, i.e., “trimming” or “cleaning” the stern bearing the female flowers by removal of larger leaves and stem material. Buds, and cleaned buds, can be used as a Cannabis plant material in practice of a method of the invention.

As used herein, “trichome” refers to a fine outgrowth or appendage on plants and certain protists. They are of diverse structure and function. Examples are hairs, glandular hairs, scales, and papillae. In reference to Cannabis, the trichome is a glandular trichome that occurs most abundantly on the floral calyxes and bracts of female plants.

As used herein, “seed” refers to an embryonic plant enclosed in a protective outer covering called the seed coat, usually with some stored food. It is a characteristic of spermatophytes (gymnosperm and angiosperm plants) and the product of the ripened ovule which occurs after fertilization and some growth within the mother plant. The formation of the seed completes the process of reproduction in seed plants (started with the development of flowers and pollination), with the embryo developed from the zygote and the seed coat from the integuments of the ovule.

As used herein, “Cannabis sativa L.” or “Cannabis sativa” refers to an annual herbaceous plant in the Cannabis genus, a species of the Cannabaceae family.

As used herein, “cannabinoid” refers to a class of diverse chemical compounds that act on cannabinoid receptors on cells that repress neurotransmitter release in the brain. These receptor proteins include the endocannabinoids (produced naturally in the body by humans and animals), the phytocannabinoids (found in Cannabis and some other plants), and synthetic cannabinoids (manufactured chemically). The most notable cannabinoid is the phytocannabinoid Δ9-tetrahydrocannabinol (THC), the primary psychoactive compound of Cannabis. Cannabidiol is another major constituent of the plant, representing up to 40% in extracts of the plant resin. There are at least 85 different cannabinoids isolated from Cannabis, exhibiting varied effects.

In certain embodiments, the cannabinoid component (which may be derived from hemp, defined as the plant Cannabis sativa L. and any part of that plant, including the seeds thereof and all derivatives, extracts, cannabinoids, isomers, acids, salts, and salts of isomers thereof) in any of the non-oral compositions described herein comprises less than about 3 wt %, less than about 2 wt %, less than about 1 wt %, less than about 0.5 wt %, less than 0.3 wt %, less than 0.2 wt %, less than 0.1 wt %, or 0 wt %, tetrahydrocannabinol (THC), the main psychoactive component of Cannabis that alters the brain function and induces changes in perception or mood of a user, based on total weight of the cannabinoid component.

In certain embodiments, the cannabinoid component in any of the non-oral composition described herein include from any of about 2 wt %, about 3 wt %, about 4 wt %, about 5 wt %, about 6 wt %, about 7 wt %, about 8 wt %, about 9 wt %, or about 10 wt % to any of about 15 wt %, about 20 wt % about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or about 100 wt %, THC, based on total weight of the cannabinoid component.

In certain embodiments, the cannabinoid component in any of the non-oral compositions described herein includes from about 2 wt % to about 10 wt %, THC, based on total weight of the cannabinoid component. In certain embodiments, the cannabinoid component in any of the non-oral compositions described herein includes from about 10 wt % to about 20 wt %, THC, based on total weight of the cannabinoid component. In certain embodiments, the cannabinoid component in any of the non-oral compositions described herein includes from about 20 wt % to about 30 wt %, THC, based on total weight of the cannabinoid component. In certain embodiments, the cannabinoid component in any of the non-oral compositions described herein includes from about 30 wt % to about 40 wt %, THC, based on total weight of the cannabinoid component. In certain embodiments, the cannabinoid component in any of the non-oral compositions described herein includes from about 40 wt % to about 50 wt %, THC, based on total weight of the cannabinoid component. In certain embodiments, the cannabinoid component in any of the non-oral compositions described herein includes from about 50 wt % to about 60 wt %, THC, based on total weight of the cannabinoid component. In certain embodiments, the cannabinoid component in any of the non-oral compositions described herein includes from about 60 wt % to about 70 wt %, THC, based on total weight of the cannabinoid component. In certain embodiments, the cannabinoid component in any of the non-oral compositions described herein includes from about 70 wt % to about 80 wt %, THC, based on total weight of the cannabinoid component. In certain embodiments, the cannabinoid component in any of the non-oral compositions described herein includes from about 80 wt % to about 90 wt %, THC, based on total weight of the cannabinoid component. In certain embodiments, the cannabinoid component in any of the non-oral compositions described herein includes from about 90 wt % to about 100 wt %, THC, based on total weight of the cannabinoid component.

In certain embodiments, the non-oral compositions described herein further include a ginger component(s). In certain embodiment, the ginger component(s) is independently present in the non-oral compositions contemplated herein in a therapeutically effective amount to treat and/or prevent nausea and/or vomiting. In certain embodiments, the ginger component(s) (e.g., one or more gingerol types, such as, without limitations, 6-gingerol, 8-gingerol, 10-gingerol, 12-gingerol) is the sole active ingredient in the non-oral compositions contemplated herein.

In certain embodiments, the amount of the ginger component(s) in a single dose of the non-oral compositions contemplated herein ranges from any of about 1 mg, about 2 mg, about 3 mg, about 4 mg, about 5 mg, about 6 mg, or about 7 mg to any of about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg. In one embodiment, the amount of ginger component(s) in a single dose of the non-oral compositions contemplated herein ranges from about 1 mg to about 20 mg. In one embodiment, the amount of ginger component(s) in a single dose of the non-oral compositions contemplated herein ranges from about 4 mg to about 15 mg. In one embodiment, the amount of ginger component(s) in a single dose of the non-oral compositions contemplated herein ranges from about 8 mg to about 12 mg.

In certain embodiments, the ginger component(s) together with the one or more cannabinoid components are present in the non-oral compositions described herein in a therapeutically effective amount to treat and/or prevent nausea and/or vomiting. In certain embodiments, the ginger component(s) (e.g., gingerol) together with one or more of the cannabinoid components (e.g., cannabidiol and/or cannabigerol) are the active ingredients in the non-oral compositions contemplated herein.

The weight to weight ratio of the one or more cannabinoid components to the ginger component(s) in the non-oral compositions contemplated herein ranges from any of about 20:1, about 19:1, about 18:1, about 17:1, about 16:1, about 15:1, about 14:1, about 13:1, about 12:1, about 10:1, about 9:1, or about 8:1 to any of about 7:1, about 6:1, about 5:1, about 4:1, about 3:1, about 2:1, or about 1:1.

In one embodiment, the weight to weight ratio of the one or more cannabinoid components to ginger component(s) in the non-oral composition described herein ranges from about 15:1 to about 1:1. In one embodiment, the weight to weight ratio of the one or more cannabinoid components to ginger component(s) in the non-oral compositions described herein ranges from about 12:1 to about 2:1. In one embodiment, the weight to weight ratio of the one or more cannabinoid components to ginger component(s) in the non-oral compositions described herein ranges from about 10:1 to about 5:1. In one embodiment, the weight to weight ratio of the one or more cannabinoid components to ginger component(s) in the non-oral compositions described herein is about 9:1. In certain embodiments, the weight to weight ratios contemplated herein refer to the total weight of all cannabinoid components in the composition relative to the total weight of all ginger components in the composition. For example, in one embodiment, the weight to weight ratios contemplated herein refer to the total weight of the cannabidiol together with the weight of the cannabigerol to the total weight of all of the ginger-derived components in the composition.

In certain embodiments, the weight to weight ratios contemplated herein refer to the weight of a single cannabinoid component in the composition relative to the total weight of all ginger components in the composition. For example, in one embodiment, the weight to weight ratios contemplated herein refer to the weight of the cannabidiol to the total weight of all of the ginger-derived materials in the composition.

In certain embodiments, the weight to weight ratios contemplated herein refer to the total weight of all cannabinoid components in the composition relative to the weight of a single ginger component in the composition. For example, in one embodiment, the weight to weight ratios contemplated herein refer to the total weight of the cannabidiol together with the weight of the cannabigerol to the weight of all of a single gingerol type in the composition.

In certain embodiments, the weight to weight ratios contemplated herein refer to the weight of a single cannabinoid component in the composition relative to the weight of a single ginger component in the composition. For example, in one embodiment, the weight to weight ratios contemplated herein refer to the weight of the cannabidiol to the weight of all of a single gingerol type in the composition.

In certain embodiments, the ginger component(s)s that may be included in the non-oral compositions contemplated herein include a ginger, a ginger root extract, a shogaol, a zingerone, a gingerol (e.g., one or more of 6-gingerol, 8-gingerol, 10-gingerol, 12-gingerol), or a combination thereof. Other ginger derived materials may also be encompassed by the term “ginger component(s)” as used herein and may also be present in the non-oral compositions contemplated herein. The weight amounts, weight ratios, and weight percentages for the ginger component(s), as contemplated herein, may refer to a single ginger component or to a plurality of ginger components together.

In one embodiment, the ginger component(s) in the non-oral compositions contemplated herein is gingerol (one type or a combination of several gingerol types). In such embodiments, the gingerol (one type or a combination of several gingerol types) amount in a single dose of the non-oral compositions described herein may range from any of about 1 mg, about 2 mg, about 3 mg, about 4 mg, about 5 mg, about 6 mg, or about 7 mg to any of about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg. In one embodiment, the amount of gingerol (one type or a combination of several gingerol types) in a single dose of the non-oral compositions described herein ranges from about 1 mg to about 20 mg. In one embodiment, the amount of gingerol (one type or a combination of several gingerol types) in a single dose of the non-oral compositions described herein ranges from about 4 mg to about 15 mg. In one embodiment, the amount of gingerol (one type or a combination of several gingerol types) in a single dose of the non-oral pharmaceutical composition described herein ranges from about 8 mg to about 12 mg.

In certain embodiments, the weight to weight ratio of the cannabidiol and/or cannabigerol (alone or in combination) to the gingerol (one type or a combination of several gingerol types) in the non-oral pharmaceutical composition described herein ranges from any of about 20:1, about 19:1, about 18:1, about 17:1, about 16:1, about 15:1, about 14:1, about 13:1, about 12:1, about 10:1, about 9:1, or about 8:1 to any of about 7:1, about 6:1, about 5:1, about 4:1, about 3:1, about 2:1, or about 1:1. In one embodiment, the weight to weight ratio of the cannabidiol and/or cannabigerol (alone or in combination) to gingerol (one type or a combination of several gingerol types) ranges from about 20:1 to about 1:1. In one embodiment, the weight to weight ratio of the cannabidiol and/or cannabigerol (alone or in combination) to gingerol (one type or a combination of several gingerol types) ranges from about 15:1 to about 2:1. In one embodiment, the weight to weight ratio of the cannabidiol and/or cannabigerol (alone or in combination) to gingerol (one type or a combination of several gingerol types) ranges from about 12:1 to about 5:1. In one embodiment, the weight to weight ratio of the cannabidiol and/or cannabigerol (alone or in combination) to gingerol (one type or a combination of several gingerol types) ranges from about 10:1 to about 7:1. In one embodiment, the weight to weight ratio of the cannabidiol and/or cannabigerol (alone or in combination) to gingerol (one type or a combination of several gingerol types) is about 9:1. In one embodiment, the weight to weight ratio of the cannabidiol and/or cannabigerol (alone or in combination) to gingerol (one type or a combination of several gingerol types) is about 8:1.

In certain embodiments, the active ingredient (e.g., the one or more cannabinoid component, the cannabidiol, the cannabigerol, the ginger component(s), or the gingerol, individually, or in any of the combinations contemplated herein) in any of the non-oral pharmaceutical compositions described herein is present at a concentration ranging from any of about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, or about 1.5 wt % to any of about 1.7 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, or about 10 wt % or any single value or sub-range therein, based on the total weight of the pharmaceutical composition.

In certain embodiments, the non-oral compositions include one or more pharmaceutically acceptable excipients, individually or in any of the combinations contemplated herein, at a concentration ranging from any of about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt %, or about 10 wt % to any of about 12 wt %, about 15 wt %, about 18 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or about 98 wt %, or any single value or sub-range therein, based on the total weight of the pharmaceutical composition.

### Pharmaceutically Acceptable Excipients

In certain embodiments, the compositions described herein may include one or more pharmaceutically acceptable excipients to arrive at a given dosage form having one or more of a target release profile, target stability, target manufacturing process, target in-vitro properties (e.g., dissolution/disintegration), target pharmacokinetic/pharmacodynamics properties, target dosing regimen, and the like.

The term “pharmaceutically acceptable excipient or carrier” refers to any inert ingredient in a composition that may act, for example, to stabilize the active ingredient. A pharmaceutically acceptable excipient can include, but is not limited to, carbohydrates, antioxidants, chelating agents, low-molecular weight proteins, high-molecular weight polymers, gel-forming agents or other stabilizers and additives. Other examples of a pharmaceutically acceptable carrier include wetting agents, emulsifying agents, surfactant and/or dispersing agents, alkalizing agents, coloring agents, synthetic dies, fillers, diluents, mineral oxides, or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

In certain embodiments, exemplary pharmaceutically acceptable excipients that may be utilized include, without limitations, acrylics, cellulose derivatives, polysaccharides, monosaccharides, gums, natural or synthetic polymers (e.g., polyalkylene oxides such as polymethylene oxides, polyethylene oxides, or polypropylene oxides, polyethylenes, polypropylenes, polyvinyl chlorides, polycarbonates, polystyrenes, polyacrylates, polycaprolactone, polymethacrylates copolymers thereof, and mixtures thereof), liposomes, disintegrants (e.g., polyvinylpyrrolidone, sodium starch glycolate, crosscarmellose sodium, or a mixture thereof), glidants, lubricants, absorption enhancers, adjuvants, surfactants, binders, softeners, plasticizers (e.g., lecithin, hydrogenated vegetable oils, glycerol ester, lanolin, methyl ester, pentaerythritol ester, rice bran wax, stearic acid, sodium potassium stearates, and the like), waxes, fats, emulsifiers, fillers, antioxidants, flavors, colorants, diluents, processing aids (e.g., granulating aids), fixing agents (e.g., polyols such as, without limitations, sorbitol, maltitol/isomalt, mannitol, starch, and the like), pH-adjusting agents, viscosity adjusting agents, solubility increasing or decreasing agents, osmotic agents, solvents, or a combination thereof.

In certain embodiments, suitable exemplary pharmaceutically acceptable excipients may include, without limitations, polyvinylpyrrolidone, natural and synthetic gums, polyvinyl alcohol, corn starch, hydrophilic and hydrophobic materials such as sustained release polymers, acrylic resins, protein-derived materials, waxes, shellacs, and solid or semi-solid oils such as hydrogenated castor oil and hydrogenated vegetable oil. More specifically, the controlled release materials can be, e.g., alkylcelluloses such as ethylcellulose, acrylic and methacrylic acid polymers and copolymers (e.g., acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid) (anhydride), methyl methacrylate, polymethacrylate, poly(methyl methacrylate), poly(methyl methacrylate) copolymer, polyacrylamide, aminoalkyl methacrylate copolymer, poly(methacrylic acid anhydride), glycidyl methacrylate copolymers, and mixtures of any of the foregoing), and cellulose ethers, such as hydroxyalkylcelluloses (e.g., hydroxypropylmethylcellulose) and carboxyalkylcelluloses. Waxes include, e.g., natural and synthetic waxes, fatty acids, fatty alcohols, and mixtures of the same (e.g., beeswax, carnauba wax, stearic acid and stearyl alcohol).

In certain embodiments, various gelling agents can be employed including, for example and without limitation, sugars or sugar derived alcohols, such as mannitol, sorbitol, and the like, starch and starch derivatives, cellulose derivatives (such as microcrystalline cellulose, sodium carboxymethyl cellulose, methylcellulose, ethyl cellulose, hydroxyethyl cellulose, hydroxypropyl cellulose, hydroxypropyl methylcellulose, cellulose esters, cellulose diesters, cellulose triesters, cellulose ethers, cellulose ester-ethers, cellulose acylates, cellulose diacylates, cellulose triacylates, cellulose acetates, cellulose diacetates, cellulose triacetates, cellulose acetate propionates, cellulose acetate butyrates, cellulose acetate succinate, cellulose acetate phthalate, hydroxypropyl methyl cellulose phthalate, hydroxy propyl methyl cellulose acetate succinate (hypermellose acetate succinate), and mixtures thereof), attapulgites, bentonites, dextrins, alginates, algenic acid salts such as sodium alginate and potassium alginate, casein, stearic acid, shellac, carrageenan, gum tragacanth, gum acacia, gum arabic, pullulan gum, dextrin, gellan gum, agar gum, tara gum, karaya, guar gum, welan gum, rhamsan gum, locust bean gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof.

In certain embodiments, various hydrophilic excipients may be included in the pharmaceutical compositions described herein. Exemplary hydrophilic excipients include, without limitations, water, low molecular weight polyols, such as, polyethylene glycol, polypropylene glycol, or a combination thereof. Examples of other suitable hydrophilic carriers include, without limitations, polyoxyethylene derivatives of a sorbitan ester, such as sorbitan monolaurate (Polysorbate 20), Polysorbate 80, Polysorbate 60, polyoxyethylene 20 sorbitan trioleate (Polysorbate 85), acetic acid, formic acid, other hydrophilic surfactants and mixtures thereof. Exemplary low molecular weight polyols include, without limitations, those having a number average molecular weight of from any of about 200 Dalton, about 400 Dalton, about 600 Dalton, about 800 Dalton, or about 1000 Dalton to any of about 2000 Dalton, about 3000 Dalton, about 4000 Dalton, about 5000 Dalton, about 6000 Da, or about 7000 Da, or any sub-range or single value therein (for instance, polyethylene glycol 400, polyethylene glycol 600, or the like).

In certain embodiments, various plasticizers may be included in the pharmaceutical compositions described herein. Suitable plasticizers may include, but not be limited to, sugar alcohol plasticizer such as triacetin, isomalt, maltitol, xylitol, erythritol, adonitol, dulcitol, pentaerythritol, or mannitol; or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

In certain embodiments, a suitable plasticizer is selected from the group consisting of phosphate esters; phthalate esters; amides; mineral oils; fatty acids and esters; fatty alcohols, vegetable oils and hydrogenated vegetable oils including acetylated hydrogenated cottonseed glyceride and acetylated hydrogenated soybean oil glycerides; acetyl tributyl citrate, acetyl triethyl citrate, Castor oil, diacetylated monoglycerides, dipropylene glycol salicylate glycerin, glyceryl cocoate, mono- and di-acetylated monoglycerides, nitrobenzene, carbon disulfide, fl-naphtyl salicylate, phthalyl glycolate, diocyl phthalate; sorbitol, sorbitol glyceryl tricitrate; sucrose octaacetate; a-tocopheryl polyethylene glycol succinate, phosphate esters; phthalate esters; amides; mineral oils; fatty acids and esters; fatty alcohols; and vegetable oils, fatty alcohols including cetostearyl alcohol, cetyl alcohol, stearyl alcohol, oleyl alcohol and myristyl alcohol; methyl abietate, acetyl tributyl citrate, acetyl triethyl citrate, diisooctyl adipate, amyl oleate, butyl ricinoleate, benzyl benzoate, butyl and glycol esters of fatty acids, butyl diglycol carbonate, butyl oleate, butyl stearate, di(beta-methoxyethyl) adipate, dibutyl sebacate, dibutyl tartrate, diisobutyl adipate, dihexyl adipate, triethylene glycol di(beta-ethyl butyrate), polyethylene glycol di(2-ethyl hexoate), diethylene glycol monolaurate, monomeric polyethylene ester, hydrogenated methyl ester of rosin, methoxyethyl oleate, butoxyethyl stearate, butyl phthalyl butyl glycolate, glycerol tributyrate, triethylene glycol dipelargonate, beta-(p-tert-amyl phenoxy)ethanol, beta(p-tert-butytphenoxy)ethanol, beta-(p-tert-butytphenoxyethyl)acetate, bis(beta-p-tert-buthylphenoxydiethyl)ether, camphor, Cumar W-1, Cumar MH-1, Cumar V-1, diamyl phthalate, (diamylphenoxy) ethanol, diphenyl oxide, technical hydroabietyl alcohol, beckolin, benzene hexahydrochlonde, Clorafin 40, Piccolastic A-5, Piccalastic A-25, Flexol B-400, Glycerol alfa-methyl alfa-phenyl ether, chlorinated naphthalene, HB-40, monoamylphthalate. Nevillac 10 o-nitrodiphenyl and Paracril 26.

Exemplary suitable coloring agents for the pharmaceutical compositions described herein may include, but not be limited to, colors such as e.g., white, black, yellow, blue, green, pink, red, orange, violet, indigo, and brown. In specific embodiments, the color of the dosage form can indicate the contents (e.g., one or more active ingredients) contained therein.

In certain embodiments, suitable excipients include, for example, diluents such as dicalcium phosphate, calcium sulfate, lactose or sucrose or other disaccharides, cellulose, cellulose derivatives, kaolin, mannitol, dry starch, glucose or other monosaccharides, dextrin or other polysaccharides, sorbitol, inositol or mixtures thereof; binders such as acacia, sodium alginate, starch, gelatin, saccharides (including glucose, sucrose, dextrose and lactose), molasses, extract of Irish moss, panwar gum, ghatti gum, mucilage of isapol husk, carboxymethylcellulose, methylcellulose, veegum, larch arabolactan, polyethylene glycols, ethylcellulose, water, alcohols, waxes, polyvinylpyrrolidone such as, e.g., PVP K90 (may be used to improve mixing of the polymer with the other ingredients) or mixtures thereof; lubricants such as talc, magnesium stearate, calcium stearate, staeric acid, hydrogenated vegetable oils, sodium benzoate, sodium chloride, leucine, carbowax 4000, magnesium lauryl sulfate, colloidal silicon dioxide and mixtures thereof, disintegrants such as starches, clays, cellulose derivatives including crosscarmellose, gums, aligns, various combinations of hydrogencarbonates with weak acids (e.g. sodium hydrogencarbonate/tartaric acid or citric acid) crosprovidone, sodium starch glycolate, agar, cation exchange resins, citrus pulp, veegum HV, natural sponge, bentonite or mixtures thereof; volatile solvents such as alcohols, including aqueous alcohols, petroleum benzine, acetone, ether or mixtures thereof; plasticizers such as sorbitol and glycerine; and others such as cocoa butter, polyethylene glycols or polyethylene oxides, e.g. with a molecular weight of about 1,000-500,000 daltons, typically about 1,000-100,000 daltons, more typically 1,000-50,000 daltons, especially about 1,000-10,000 daltons, in particular about 1,500-5,000 daltons, and mixtures thereof, hydrogenated vegetable oils, glycerinated gelatin or mixtures thereof.

In certain embodiments, suitable antioxidants may include BHA, BHT, t-butyl hydroquinone, calcium ascorbate, gallic acid, hydroquinone, maltol, octyl gallate, sodium bisulfate, sodium metabisulfite, tocopherol and derivates thereof, citric acid, tartaric acid, and ascorbic acid. Other antioxidants include trivalent phosphorous such as phosphite, phenolic antioxidants, hydroxylamines, and lactones such as substituted benzofuranones. Hindered phenols, thiosynergists and/or hindered amines are useful for the long-term stability for polymers, whereas the following antioxidants are suitable for use also in situation where the active substance is subject to oxidation: acids (ascorbic acid, erythorbic acid, etidronic acid, gallic acid, hypophosphorous acid, nordihydroguairetic acid, propionic acid etc.), phenols (e.g. BHA, BHT, t-butyl hydroquinone, dodecyl gallate, octyl gallate, 1,3,5-trihydroxybenzene), organic and inorganic salts (calcium ascorbate, sodium ascorbate, sodium bisulphite, sodium metabisulfite, sodium sulfite, potassium bisulphite, potassium metabisulphite), esters (calcium ascorbate, dilauryl thiodipropionate, dimyristyl thiodipropionate, distearyl thiodipropionate), pyranon (maltol), and vitamin E (tocopherol, D-α-tocopherol, DL-α-tocopherol, tocopherol acetate, d-α-tocopheryl acetate, dl-α-tocopheryl acetate.

In certain embodiments, suitable antioxidants may include, without limitations, sterically hindered phenols, aryl amines, thioureas, thiocarbamates, phosphites, thioether esters, and combinations of the foregoing. Other suitable examples of antioxidants include, but are not limited to, alkylated monophenols, including but not limited to, 2,6-di-tert-butyl-4-methylphenol, 2-tert-butyl-4,6-di-methylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which are linear or branched in the side chains, for example, 2,6-di-nonyl-4-methylphenol, 2,4-dimethyl-6-(1′-methylundec-1′-yl)phenol, 2,4-dimethyl-6-(1′-methylheptadec-1-yl)phenol, 2,4-dimethyl-6-(1′-methyltridec-1-yl)phenol and mixtures thereof, alkylthiomethylphenols, including but not limited to, 2,4-dioctylthiornethyl-6-tert-hutylphenol, 2,4-dioctylthiomethyl-6-methylphenol, 2,4-dioetylthiomethyl-6-ethylphenol, 2,6-di-dodecylthiornethyl-4-nonylphenol, hydroquinones and alkylated hydroquinones, including but not limited to, 2,6-di-tert-hutyl-4-methoxyphenol, 2,5-di-tert-butylhydroquinone, 2,5-di-tort-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxyphenyl) adipate, tocopherols, including but not limited to, α-tocopherol, β-tocopherol, γ-tocopherol, δ-tocopherol and mixtures thereof (vitamin E), hydroxylated thiodiphenyl ethers, including but not limited to, 2,2′-thiobis(6-tort-butyl-4-methylphenol), 2,2′-thiobis(4-oetylphenol), 4,4′-thiobis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)-disulfide, alkylidenebisphenols, including but not limited to, 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)-phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-test-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis(5-tert-butyl-4-hydroxy-2-methylphenyl)-4-n-dodecylmercaptobutane, 1,5,5-tetra-(5-tert-butyl-4-hydroxy-2-methylphenyl)pentane, O-, N- and S-benzyl compounds, including but not limited to, 3,5,3′,5′-tetra-tert-butyl.-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5-di-tert-butyl-4-hydroxybenzylmercaptoacetate, hydroxybenzylated malonates, including but not limited to, dioctadecyl-2,2-bis(3,5-di-tert-butyl-2-hydroxybenzyl)malonate, di-octadecyl-2-(3-tert-butyl-4-hydroxy-5-methylbenzyl)malonate, didodecylmercaptoethyl-2,2-bis(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate, aromatic hydroxybenzyl compounds, including but not limited to, 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol, triazine compounds, including but not limited to, 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris-(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris-(3,5-di-tert-butyl-4-hydroxyphenylethyl)-1,3,5-triazine, 1,3,5-tris(3,5-di-tert-butyl-4-hydroxy-phenylpropionyl)-hexahydro-1,3,5-triazine, 1,3,5-tris(3,5-dicyclohexyl-4-hydroxybenzyl)iso-cyanurate, benzylphosphonates, including but not limited to, dimethyl-2,5-di-tert-butyl-4-hydroxybenzylphosphonate, diethyl-3,5-di-tert-butyl-4-hydroxybenzylphosphonate, dioctadecyl3,5-di-tent-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid, acylaminophenols, including but not limited to, 4-hydroxylauranilide, 4-hydroxystearanilide, octyl N-(3,5-di-tert-butyl-4-hydroxyphenyl)carbamate, esters of β-(3,5-di-tert-butyl-4-hydroxyphenyl)propionic acid with mono- or polyhydric alcohols, e.g. with methanol, ethanol n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, esters of β-(5-tert-butyl-4-hydroxy-3-methylphenyl)propionic acid with mono- or polyhydric alcohols, e,g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis-(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2] octane; 3,9-bis[2-{3-(3-tert-butyl-4-hydroxy-5-methylphenyl)propionyloxy}-1,1-dimethylethyl]-2,4,8,10-tetraoxaspiro[5.5]-undecane, esters of 6-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2,2]octane, esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycal, thiodiethyl.ene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, amides of 6-(3,5-di-tert-butyl-4-hydroxyphenyl)propionic acid e.g. N,N′-bis(3,5-di-tert-butylA-hydroxyphenylpropionyl)hexamethylenediamide, N,N′-bis(3,5-di-tert-butyl-4-hydroxyphenylpropionyl)trimethylenediamide, N,N′-bis(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide (Naugard®XL-1, supplied by Uniroyal), ascorbic acid (vitamin C), aminic antioxidants, including but not limited to, N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyI)-N′-phenyl-p-phenylenediamine, N-cyclohexyl-N′-phenyl-p-phenyienediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octylated diphenylamine, including but not limited to, p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis(4-methoxyphenyl)amine 2,6-di-tert-butyl-4-dimethylaminomethylphenol, 2,4′-diaminodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyldiphenylamines, a mixture of mono- and dialkylated teak-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octyl-phenothiazines, N-allylphenothiazine, N,N,N′,N′-tetraphenyl-1,4-diaminobut-2-ene, and combinations of the foregoing.

In certain embodiments, the pharmaceutical compositions may include alkalizing agent(s), such as, without limitations, magnesium oxide, ammonium hydroxide, sodium hydroxide, sodium carbonate, sodium citrate, trisodium phosphate and/or disodium phosphate.

In certain embodiments, the pharmaceutical compositions may include lubricant(s)/release agent(s) such as, but not limited to, fatty acids and their salts, fatty alcohols, fatty esters, fatty amines, fatty amine acetates and fatty amides. Other suitable lubricants may include, but not be limited to, glyceryl behenate (Compritol™ 888), metallic stearates (e.g., magnesium, calcium and sodium stearates), stearic acid, hydrogenated vegetable oils (e.g., Sterotex™), talc, waxes such as beeswax and carnauba wax, silica, fumed silica, colloidal silica, calcium stearate, long chain fatty alcohols, boric acid, sodium benzoate and sodium acetate, sodium chloride, DL-Leucine, polyethylene glycols (e.g., Carbowax™ 4000 and Carbowax™ 6000), sodium oleate, sodium benzoate, sodium acetate, sodium lauryl sulfate, sodium stearyl fumarate (Pruv™), magnesium lauryl sulfate, stearic acid, stearyl alcohol, mineral oil, paraffin, micro crystalline cellulose, glycerin, propylene glycol and combinations thereof.

In certain embodiments, the pharmaceutical compositions may include diluents such as, but not limited to, lactose USP, lactose USP (anhydrous), lactose USP (spray dried), starch USP, directly compressible starch, mannitol USP, sorbitol, dextrose monohydrate, microcrystalline cellulose NF, dibasic calcium phosphate dihydrate NF, sucrose-based diluents, confectioner's sugar, monobasic calcium sulfate monohydrate, calcium sulfate dihydrate NF, calcium lactate trihydrate granular NF, dextrates NF (e.g., Emdex™), dextrose (e.g., Cerelose™), inositol, hydrolyzed cereal solids such as the Maltrons™ and Mor-Rex™, amylose, powdered cellulose (e.g., Elcema™), calcium carbonate, glycine, bentonite, polyvinylpyrrolidone, and the like.

In certain embodiments, the pharmaceutical compositions may include oils and fats such as, but not be limited to, almond oil, argan oil, avocado oil, canola oil, cashew oil, castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, walnut oil, and watermelon seed oil. Other oil and fats that may be in the fill of the PVA shell may include, but not be limited to, fish oil (omega-3), crill oil, animal or vegetable fats, e.g., in their hydrogenated form, mono-, di-, and tri-glycerides with C₁₂-, C₁₄-, C₁₆-, C₁₈-, C₂₀- and C₂₂-fatty acids.

In certain embodiments, the pharmaceutical compositions may include pH modifiers such as, but not be limited to, hydrochloric acid, potassium hydroxide, sodium hydroxide, ammonium hydroxide, sulfuric acid, phosphoric acid, and nitric acid.

In certain embodiments, the pharmaceutical compositions may include other exemplary excipients such as, but not be limited to, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids, polyacrylamides, and polyacrylic acid esters, polymethacrylic acids, polymethacrylamides, and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; and other similar polymers; inorganic fillers, such as the oxides of magnesium aluminum, silicon, titanium, etc.

In certain embodiments, the pharmaceutical compositions may include other pharmaceutically acceptable excipients such as, without limitations, a hydrophobic material, including, but not limited to, digestible, long chain (C₈-C₅₀, especially C₁₂-C₄₀), substituted or unsubstituted hydrocarbons, such as natural or synthetic waxes (such as beeswax, glycowax, castor wax and carnauba wax), fatty alcohols (such as lauryl, myristyl, stearyl, cetyl or preferably cetostearyl alcohol), fatty acids, including, but not limited to, mono-diglyceride of medium chain fatty acids (such as caprylic, capric, caproic, lauric, oleic, linoleic), medium chain triglycerides, fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones.

In certain embodiments, the pharmaceutical compositions may include other pharmaceutically acceptable excipients such as, without limitations, polyvinyl alcohols, polyvinyl pyrrolidone, polyalkylene oxides, polyacrylic acid, cellulose, cellulose ethers, cellulose esters, cellulose amides, polyvinyl acetates, polycarboxylic acids and salts, acetic acid, caprylic acid, oleic acid, polyaminoacids or peptides, polyamides, polyacryl amide, copolymers of maleic/acrylic acids, polysaccharides including starch and gelatin, natural gums such as xanthan, and carrageenans. For example, polymers can be selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and combinations thereof, or selected from polyvinyl alcohols, polyvinyl alcohol copolymers and hydroxypropyl methyl cellulose (HPMC), methacrylic acid/methyl methacrylate, methacrylic acid/ethyl acrylate copolymers, methacrylic acid/methyl acrylate/methyl methacrylate copolymers, shellac, hydroxypropyl methylcellulose phthalate, hydroxyl propyl methyl cellulose acetate succinate, hydroxypropyl methyl cellulose trimellitate, cellulose acetate phthalates, polyvinyl acetate phthalates, PEG-35 castor oil, caprylocaproyl polyoxyl-8 glycerides, glyceryl distearate, and combinations thereof.

In certain embodiments, the pharmaceutical compositions may include high HLB surfactants such as, without limitations, polysorbate 80-polyoxyethylene (20) sorbitan monooleate, polyoxyl 40 hydrogenated castor oil, polyoxyl 35 castor oil, caprylocaproyl macrogol glycerides, and combinations thereof.

In certain embodiments, the pharmaceutical compositions may include fillers such as, without limitations, lactose, microcrystalline cellulose, and combinations thereof.

Other pharmaceutically acceptable excipients may also be utilized as recognized by those skilled in the art.

### Release Profile

In certain embodiments, any of the non-oral compositions described herein may be formulated to have a target release profile, such as, without limitations, controlled release, immediate release, first order release profile, zero order release profile, pulsatile release, or any combination thereof. In certain embodiments, any of the non-oral compositions described herein may be coated with a suitable coating (e.g., to attain a certain release profile) and/or with a cosmetic coating.

In certain embodiments, the non-oral compositions described herein have an immediate release profile. The term “immediate release,” as used herein refers to a non-oral composition releasing at least about 85%, at least about 90% or at least about 95% of the active agent within 15 minutes, within 30 minutes, within 45 minutes, or within 60 minutes as measured by in-vitro dissolution in a USP Apparatus 2 (basket) or in a USP Apparatus 2 (paddle) or in a USP Apparatus 3 (reciprocating cylinder) or in a USP Apparatus 4 (flow-through cell system) suitable conditions as recognized by those skilled in the art in accordance with industry guidelines provided by a given regulatory authority.

In certain embodiments, the non-oral compositions described herein have a controlled release profile. The term “controlled release,” as used herein refers to a non-oral composition releasing the active agent over a period of time, e.g., to provide a once daily or twice daily dosage form.

In certain embodiments, the non-oral compositions described herein have a zero order release rate, such that the release rate of the active agent is constant over a period of time.

In certain embodiments, the non-oral compositions described herein have a first order release rate, such that the release rate of the active agent is proportional to the concentration of the active agent in the pharmaceutical composition.

### Method of Treatment

In certain embodiments, the instant disclosure relates to a method for treating subjects in need thereof with any of the pharmaceutical compositions described herein. The subject in need thereof may be a cancer subject that is either experiencing nausea and/or vomiting or is being treated prophylactically against nausea and/or vomiting that may be developed as a result of the cancer treatment regime, the cancer itself, other drugs, and other nausea and/or vomiting causes as understood by one skilled in the art. In certain embodiments, the subject in need thereof may be a subject experiencing physiological nausea and/or vomiting that may be triggered by peripheral factors such as ingestion of toxins or consumption of toxins through other means (e.g., alcohol/drug intoxication), disturbance of the vestibular system, peritoneal inflammation, bowel obstruction, or other underlying medical conditions. In certain embodiments, the subject in need thereof may be a subject experiencing nausea and/or vomiting related to disorders of delayed gastric emptying as, for example, diabetes and idiopathic gastroparesis. In certain embodiments, the subject in need thereof may be a subject experiencing nausea and/or vomiting that is psychogenic and may be triggered by anxiety, threatening situation, a situation that is regarded as distasteful by the subject, by the subject's hostility (such as a temper tantrum), or another psychological trigger. In certain embodiments, the subject in need thereof may be a subject experiencing the nausea and/or vomiting that may be induced by cytotoxic chemotherapy and/or radiotherapy. In certain embodiments, the subject in need thereof may be a subject experiencing nausea and/or vomiting that may be post-operative and may be attributed to the anesthetic agents and/or the analgesic agents that are administered to the subject. In certain embodiments, the subject in need thereof may be a subject that is experiencing nausea and/or vomiting related to motion sickness. In certain embodiments, the subject in need thereof may be a subject experiencing nausea and/or vomiting that is pregnancy related. Any other subject who experiences (or may experience) nausea and/or vomiting and/or symptoms related to nausea and/or vomiting may also engage in the methods of treatments contemplated herein.

In certain embodiments, the pharmaceutical compositions described herein may be administered to the subject in need thereof via one or more of the following administration routes: parenteral, rectal, vaginal, topical, transdermal, ocular, nasal, or otic.

In certain embodiments, administering includes injecting the pharmaceutical composition to the subject in need thereof. In certain embodiments, injecting may be done as an intramuscular injection, as a subcutaneous injection, as an intravenous injection, or as an intradermal injection.

In certain embodiments, administering includes placing or applying the pharmaceutical composition in or proximate to a bodily cavity (e.g., for rectal or vaginal administration). In certain embodiments, administering may include placing or inserting a suppository, a tablet, an enema, or a pessary into a bodily cavity (such as the rectum, the vagina, or the urethra). In certain embodiments, placing or inserting may be done manually or with a tool, such as an applicator, forceps, and the like.

In certain embodiments, administering includes causing the subject to apply the composition or to place the composition in or proximate to a bodily cavity (such as the rectum, the vagina, or the urethra). In certain embodiments, the composition that is applied or placed in or proximate to the bodily cavity is to remain undisturbed while the composition disintegrates and/or dissolves.

In certain embodiments, the composition may remain undisturbed in the bodily cavity of the subject for a duration sufficient to deliver a therapeutically effective amount of the active ingredient to alleviate, minimize, treat, and/or prevent the occurrence of nausea and/or vomiting.

In certain embodiments, administering includes applying the pharmaceutical composition (e.g., an ointment, cream, gel, spray, patch, foam, or the like) onto the skin of a subject in need thereof. As used herein, the terms “application,” “apply,” and “applying” with respect to a disclosed pharmaceutical composition being suitable for topical or transdermal administration, refer to any manner of administering a topical composition to the skin of a patient which, in medical practice, delivers the composition to the patient's skin surface. Smearing, rubbing, spreading, spraying a disclosed topical composition, with or without the aid of suitable devices, on a patient's skin are all included within the scope of the term “application,” as used herein. The terms “topical” or “topically” with respect to administration or application of a disclosed formulation refer to epicutaneous administration or application, or administration onto skin.

In certain embodiments, the pharmaceutical composition may be administered once a week, once every three days, every other day, once a day, twice a day, three times a day, four times a day, or on an as needed basis.

In certain embodiments, the compositions described herein may be administered concurrently, simultaneously, or sequentially along with other drugs as part of the treatment regimen for a given condition. In certain embodiments, the pharmaceutical composition may be administered to a subject concurrently, simultaneously, or sequentially along with other drugs as part of a treatment regimen for a given condition. In one embodiment, the compositions described herein may be administered to a subject in need thereof concurrently, simultaneously, or sequentially along with drugs prescribed as part of a cancer treatment regimen.

The term “concurrently” as used herein means that a dose of one agent (e.g., the non-oral pharmaceutical compositions contemplated herein) is administered prior to the end of the dosing interval of another agent (e.g., cancer treatment active agent).

The term “simultaneously” as used herein means that a dose of one agent (e.g., the non-oral pharmaceutical compositions contemplated herein) is administered approximately at the same time as another agent (e.g., cancer treatment active agent) regardless of whether the agents are administered separately via the same or different routes of administration or in a single pharmaceutical composition or dosage form.

The term “sequentially” as used herein means that a dose of one agent (e.g., the non-oral pharmaceutical compositions contemplated herein) is administered first and thereafter a dose of another agent is administered second (e.g., cancer treatment active agent). The subsequent administration of the second agent may be inside or outside the dosing interval of the first agent.

### Method of Manufacturing

In certain embodiments, the instant disclosure relates to a method for manufacturing any of the pharmaceutical compositions. Generally, the method includes combining one or more cannabinoid components with one or more ginger components and a pharmaceutically acceptable excipient to form the pharmaceutical composition. A variety of methods may be used to combine these components, some of which are described in further detail below. The below description should not be construed as limiting as other suitable methods may also be implemented.

In certain embodiments, the instant disclosure relates to methods of manufacturing pharmaceutical compositions formulated for one or more of parenteral administration, rectal administration, vaginal administration, topical administration, transdermal administration, ocular administration, otic administration, or nasal administration.

In certain embodiments, the instant disclosure may be directed to methods of manufacturing pharmaceutical compositions in a form of a solution, suspension, emulsion, tablet, suppository, ointment, cream, gel, lotion, spray, patch, foam, pessary, drops, powder, implant, insert, or a combination thereof.

Exemplary manufacturing procedures include, without limitations, compression (e.g., to form a compressed tablet and optionally a compression coating), granulation (e.g., wet granulation or dry granulation), tableting, spray drying, freeze drying, extrusion, rotary die encapsulation, blending, milling, mixing, autoclaving, sterilizing, compaction, coating, packaging, combinations thereof, and the like. The skilled artisan would recognize the various manufacturing steps to be taken to arrive at a given dosage form.

The prepared pharmaceutical compositions may subsequently be packaged into a suitable container. Suitable containers include, without limitations, a bottle, a bag, a blister package, wrappers, or any other suitable packaging. In certain embodiments, the instant disclosure is also directed to a kit that includes a container and any of the pharmaceutical compositions described herein stored within the container. In certain embodiments, the kit may also include a delivery device (e.g., an applicator or a tool to assist with administration of the pharmaceutical composition).

### EXAMPLES

The following prophetic examples are set forth to assist in understanding the invention and should not be construed as specifically limiting the invention described and claimed herein. Such variations of the invention, including the substitution of any or all equivalents now known or later developed, which would be within the purview of those skilled in the art, and changes in formulation or minor changes in therapeutic design, are to be considered to fall within the scope of the invention incorporated herein.

Table 1 below depicts an exemplary prophetic formulation of a suppository composition according to certain embodiments.

TABLE 1
Exemplary Formulation of a Suppository Composition

| | | |
|---|---|---|
| Gelatin | 35.0-70.0 | wt % |
| Glycerin | 3.0-15.0 | wt % |
| Active Ingredients (e.g., one or more of | 0.5-10.0 | wt % |
| cannabidiol, cannabigerol, and gingerol) | | |
| Water | 5.0-61.5 | wt % |

Table 2 below depicts an exemplary prophetic formulation of a transdermal composition according to certain embodiments.

TABLE 2
Exemplary Formulation of a Transdermal Composition

| | | |
|---|---|---|
| Levulinic Acid | 5.0-15.0 | wt % |
| Polyvinylpyrrolidone | 5.0-15.0 | wt % |
| Active Ingredients (e.g., one or more of | 0.5-20.0 | wt % |
| cannabidiol, cannabigerol, and gingerol) | | |
| Polyacrylate | 40.0-60.0 | wt % |

The composition is then included in a transdermal delivery device.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A pharmaceutical composition comprising:
one or more cannabinoid components in an effective amount to treat nausea and/or vomiting;
a ginger component; and
a pharmaceutically acceptable excipient,
wherein the pharmaceutical composition is formulated for non-oral administration.

2. The pharmaceutical composition of claim 1, wherein the one or more cannabinoid components comprises cannabidiol, cannabigerol, tetrahydrocannabinol, cannabinol, cannabichromene, or a combination thereof.

3. (canceled)

4. (canceled)

5. The pharmaceutical composition of claim 2, wherein the w/w ratio of the cannabidiol to the cannabigerol ranges from about 15:1 to about 1:1, from about 12:1 to about 2:1, from about 10:1 to about 5:1, or about 8:1.

6. The pharmaceutical composition of claim 1, wherein the ginger component comprises gingerol.

7. The pharmaceutical composition of claim 1, wherein the w/w ratio of the one or more cannabinoid components to the ginger component ranges from about 15:1 to about 5:1, from about 12:1 to about 7:1, from about 10:1 to about 8:1, or about 9:1.

8. (canceled)

9. The pharmaceutical composition of claim 1, wherein the pharmaceutical composition is suitable for administration via one or more of: parenteral route, rectal route, vaginal route, topical route, transdermal route, ocular route, otic route, or nasal route.

10. A pharmaceutical composition comprising:
one or more cannabinoid components;
a ginger component; and
a pharmaceutically acceptable excipient;
wherein the one or more cannabinoid components and the ginger component, together, are present in the pharmaceutical composition in an effective amount to treat nausea and/or vomiting, and
wherein the pharmaceutical composition is formulated for non-oral administration.

11. The pharmaceutical composition of claim 1, wherein the pharmaceutical composition comprises less than 0.3 wt % THC (delta-9-tetrahydrocannabinol), based on total weight of the one or more cannabinoid components.

12. A pharmaceutical composition comprising:
an effective amount of cannabidiol to treat nausea and/or vomiting;
a cannabigerol;
a ginger component; and
a pharmaceutically acceptable excipient,
wherein the pharmaceutical composition is formulated for non-oral administration.

13. A method for treating nausea and/or vomiting, the method comprising:
administering to a subject in need thereof a pharmaceutical composition comprising one or more cannabinoid components and a ginger component, wherein the administering is done via a non-oral route.

14.-23. (canceled)

24. The method of claim 13, wherein the one or more cannabinoid components is present in the pharmaceutical composition in an effective amount to treat nausea and/or vomiting.

25. The method of claim 13, wherein the one or more cannabinoid components and the ginger component, together, are present in the pharmaceutical composition in an effective amount to treat nausea and/or vomiting.

26.-28. (canceled)

29. A method for preparing a pharmaceutical composition according to claim 1, the method comprising:
combining the one or more cannabinoid components, the ginger component and the pharmaceutically acceptable excipient to form the pharmaceutical composition.

30. (canceled)

31. A method for preparing a pharmaceutical composition according to claim 12, the method comprising:
combining the effective amount of cannabidiol, the cannabigerol, the ginger component and the pharmaceutically acceptable excipient to form the pharmaceutical composition.

32. (canceled)

33. The pharmaceutical composition of claim 1, wherein the ginger component comprises ginger, a ginger root extract, a shogaol, a zingerone, a gingerol, or a combination thereof.

34. The pharmaceutical composition of claim 12, wherein the ginger component comprises ginger, a ginger root extract, a shogaol, a zingerone, a gingerol, or a combination thereof.

35. The pharmaceutical composition of claim 1, wherein the composition is in a form of a tablet, capsule, spray formulation, an ointment, cream, lotion, gel, foam, solution, emulsion, suspension, suppository, patch, pessary, drops, powder, implant, insert or a combination thereof.

36. The pharmaceutical composition of claim 1, wherein the amount of one or more cannabinoid components is about 5 mg to about 150 mg, about 10 mg to about 145 mg, about 15 mg to about 140 mg, about 20 mg to about 135 mg, about 25 mg to about 130 mg, about 30 mg to about 125 mg, about 35 mg to about 120 mg, about 40 mg to about 115 mg, about 45 mg to about 110 mg, about 50 mg to about 105 mg, about 55 mg to about 100 mg, about 60 mg to about 95 mg, about 65 mg to about 90 mg, about 70 mg to about 85 mg, or about 75 mg to about 80 mg in a single dose of the pharmaceutical composition.

37.-44. (canceled)

45. The pharmaceutical composition of claim 1, wherein the amount of the ginger component is from about 1 mg to about 20 mg, about 4 mg to about 15 mg, or about 8 mg to about 12 mg in a single dose.

46.-54. (canceled)

55. A kit comprising a container and a pharmaceutical composition of claim 1, wherein the container comprises a bottle, a bag, a blister package, a tube, a delivery device, or a device for assisting with suppository placement.
